(19) United States
(12) Patent Application Publication
(10) Pub. No.: US 2018/0036303 A1
(43) Pub. Date: Feb. 8, 2018

(54) LSD FOR THE TREATMENT OF ALZHEIMER'S DISEASE

(71) Applicant: Eleusis Benefit Corporation, PBC, New York, NY (US)

(72) Inventor: Shlomi RAZ, Brooklyn, NY (US)

(21) Appl. No.: 15/556,459

(22) PCT Filed: Mar. 10, 2016
(86) PCT No.: PCT/US16/21766

§ 371 Date: Sep. 7, 2017

Related U.S. Application Data
(60) Provisional application No. 62/131,000, filed on Mar. 10, 2015.

(51) Int. Cl. A61K 31/48; A61K 31/13; A61K 45/06

(52) U.S. Cl. CPC A61K 31/48; A61K 45/06; A61K 31/13

(57) ABSTRACT

The invention features methods and compositions for the treatment of Alzheimer's Disease using lysergic acid diethylamide and pharmaceutically acceptable salts thereof.

### BACKGROUND OF THE INVENTION

This invention relates to the use of LSD for the treatment of Alzheimer's disease.

Alzheimer's disease (hereinafter “Alzheimer's disease” or “AD”) is a neurodegenerative disease and the most common cause of dementia. This disease manifests as a gradual but progressive decline in memory, thinking skills and behavior that is accelerated relative to normal aging (Reitz et al. 2011 Nat Rev Neurol 7: 137-152). Eventually, patients are unable to recognize familiar people or carry out the simplest task. Alzheimer's disease is, at this time, among the leading causes of death in the United States (US). There are two predominant forms of the disease: Familial Alzheimer's disease is typically caused by dominant genetic mutations. This form of the disease is a rare and devastating illness with onset occurring in mid-life. The second and far more common form of the disease is Sporadic or Late onset Alzheimer's disease.

The onset of Alzheimer's disease typically occurs after the age of 62 years. As the world population and human longevity increase, so do the numbers of people affected by Alzheimer's disease globally. The estimated worldwide costs of dementia, of which Alzheimer's disease accounts for up to 80% of cases, was US$604 billion in 2010, which was greater than 1% of US GDP (VVimo and Prince 2010 World Alzheimer Report 2010: The Global Economic Impact of Dementia 1-93). The cost of caring for Alzheimer patients in the US is expected to increase from US$172 million in 2010, to US$1.07 trillion in 2050 (Alzheimer's Association. “Changing the Trajectory of Alzheimer's Disease: A National Imperative (2010)”).

At this time, the few drugs that are approved for treatment of this disease provide some symptomatic relief, but this is typically of relatively short duration, and the therapies do not alter the course of disease progression (Alzheimer's Association. “Changing the Trajectory of Alzheimer's Disease: A National Imperative (2010)”). Therapies that delay the onset of the disease, reduce the rate of disease progression, or that can do both are urgently needed. Therapies that can achieve either of these goals will reduce the number of individuals with disease, or reduce the number of individuals with the more advanced and debilitating stages of disease (Brookmeyer et al. 2007 Alzheimers Dement 3: 186-191). It is projected that if the onset of Alzheimer's disease is delayed by 5 years due to availability of a breakthrough therapy in 2015, 43% of the 13.5 million Americans expected to have the condition in 2050 would not have the disease, and there will be fewer people with advanced disease.

The principal risk factor for Alzheimer's disease is age, and prevalence of the disease increases with age (approximately 10% of individuals over 65 and approximately 50% of individuals over 85). The incidence of the disease doubles every 5 years after 65 years of age, with the diagnosis of about 1,275 new cases per year per 100,000 persons older than 65 years of age (Querfurth et al., 2010 NEJM 362:4). Both men and women are affected by Alzheimer's disease, but women generally represent a higher percentage of cases overall (roughly 60% to 40%), possibly due to greater longevity. People suffering from Alzheimer's disease tend to live approximately 3 to 9 years after diagnosis, on average.

In view of the fact that more than 4.5 million people in the United States alone suffer from Alzheimer's disease (and this number will continue to grow as the population ages), the cruel and unforgiving degenerative and debilitative nature of Alzheimer's disease as it develops, and the high costs associated with the care for people suffering from Alzheimer's disease, there is a real and immediate need for an effective medical therapy that can ameliorate the symptoms, or delay the onset, of Alzheimer's disease.

### SUMMARY OF THE INVENTION

The invention features a method of treating Alzheimer's disease in a subject, the method including administering to the subject a pharmaceutical composition comprising lysergic acid diethylamide, or a salt thereof, (LSD) in an amount sufficient to treat the Alzheimer's disease. In particular embodiments, the pharmaceutical composition is a unit dosage form including from 2 to 30 μg of lysergic acid diethylamide or a pharmaceutically acceptable salt thereof (e.g., 25±5, 15±5 μg, 12.5±5 μg, 10±2 μg, 8±2 μg, 7.5±2.5 μg, 6±2 μg, or 4±2 μg of lysergic acid diethylamide or a pharmaceutically acceptable salt thereof). In certain embodiments, the method includes reducing agitation, reducing apathy, reducing aggression, reducing irritability, delaying the onset of agitation, delaying the onset of apathy, delaying the onset of aggression, improving cognitive function, reducing the severity of an AD-associated neuropsychiatric condition, delaying the loss of cognitive function, or delaying the onset of an AD-associated neuropsychiatric conditions in the subject. The methods of the invention can include reducing agitation, reducing apathy, reducing irritability, or reducing aggression in a subject having Alzheimer's disease with comorbid dementia; reducing agitation, reducing apathy, or reducing aggression in a subject having Alzheimer's disease with mild cognitive impairment due to Alzheimer's disease; reducing agitation, reducing apathy, reducing irritability, or reducing aggression in a subject having Alzheimer's disease with asymptomatic Alzheimer's disease; or reducing agitation, reducing apathy, reducing irritability, or reducing aggression in a subject having Alzheimer's disease with prodromal Alzheimer's disease. The methods of the invention can include improving memory in the subject, improving learning capacity in the subject, delaying the loss of memory in the subject, delaying the loss of learning capacity in the subject, reducing the severity of dementia in the subject, delaying the onset of dementia in the subject, reducing the severity of depression in the subject, delaying the onset of depression in the subject, reducing the severity of anxiety in the subject, and/or delaying the onset of anxiety in the subject. For example, the method can include delaying the loss of cognitive function or delaying the onset of dementia in a subject with mild cognitive impairment due to Alzheimer's disease; improving cognitive function or reducing the severity of dementia in a subject having Alzheimer's disease with comorbid dementia; improving cognitive function or reducing the severity of depression in a subject having Alzheimer's disease with comorbid depression; and/or improving cognitive function or reducing the severity of anxiety in a subject having Alzheimer's disease with comorbid anxiety. In certain embodiments, the method can include delaying the loss of cognitive function or reducing the severity of an AD-associated neuropsychiatric condition in a subject having Alzheimer's disease with comorbid dementia; delaying the loss of cognitive function or delaying the onset of an AD-associated neuropsychiatric in a subject with mild cognitive impairment due to Alzheimer's disease; delaying the loss of cognitive function or delaying the onset of an AD-associated neuropsychiatric condition in a subject with asymptomatic Alzheimer's disease; or delaying the loss of cognitive function or delaying the onset of an AD-associated neuropsychiatric condition in a subject with prodromal Alzheimer's disease. In particular embodiments, the AD-associated neuropsychiatric condition is depression, anxiety, agitation, apathy, irritability, and/or aggression. In other embodiments, the cognitive function is memory and/or learning. The invention can include delaying the loss of cognitive function or delaying the onset of dementia in a subject with mild cognitive impairment due to Alzheimer's disease; delaying the loss of cognitive function or delaying the onset of dementia in a subject with asymptomatic Alzheimer's disease; or delaying the loss of cognitive function or delaying the onset of dementia in a subject with prodromal Alzheimer's disease. In some embodiments, the lysergic acid diethylamide, or a pharmaceutically acceptable salt thereof, is administered in a dosing regimen from once daily to once weekly. In certain embodiments, the dosing regimen is once every three, four, or five days. The dosing regimen can include administering to the subject an average of from 8 μg to 90 μg lysergic acid diethylamide, or a pharmaceutically acceptable salt thereof, per week (e.g., from 8 μg to 25 μg, from 15 μg to 35 μg, or from 30 μg to 75 μg per week). In particular embodiments, the pharmaceutical composition is formulated for sustained release. In still other embodiments, the pharmaceutical composition is formulated for immediate release. The treatments of the invention can be administered in conjunction with one or more additional treatments for Alzheimer's disease. For example, the method can further include administering to the subject a second agent, such as a neuronal growth factor, a neuronal survival factor, a neuronal trophic factor, a cholinergic modulator, an adrenergic modulator, a nonadrenergic modulator, a dopaminergic modulator, a glutaminergic modulator or an agent that modulates PKC, PKA, GABA, NMDA, cannabinoid, AMPA, kainite modulator, phosphodiesterase (PDE), CREB or nootropic pathways, a cholinomimetic agent or N-methyl-D-aspartate receptor (NMDA) antagonist, within 1-30 days of administering lysergic acid diethylamide or a pharmaceutically acceptable salt thereof. As a combination therapy, the lysergic acid diethylamide or a pharmaceutically acceptable salt thereof and second agent can be formulated and administered together or separately, each active administered in an amount that together is sufficient for the treatment of Alzheimer's disease. When the second agent is a cholinomimetic agent, the cholinomimetic agent can be selected from inhibitors of acetylcholine degradation, inducers of acetylcholine synthesis, acetylcholine agonists, and muscarinic M2-receptor antagonists. In particular embodiments, the cholinomimetic agent is an acetylcholinesterase inhibitor. When the second agent is an NMDA antagonist, the NMDA antagonist can be, for example, memantine or a pharmaceutically acceptable salt thereof.

For use in the methods of the invention, the LSD, or a salt thereof, can be administered systemically, including, for example, by intravenous, intramuscular, or subcutaneous injection, orally, sublingually, by inhalation, or by transdermal application.

The term “administration” or “administering” refers to a method of giving a dosage of a pharmaceutical composition to a patient, where the method is systemic, e.g., oral, topical, transdermal, by inhalation, intravenous, intraperitoneal, intracerebroventricular, intrathecal, or intramuscular. The preferred method of administration can vary depending on various factors, e.g., the components of the pharmaceutical composition, site of administration, and severity of the symptoms being treated.

As used herein, the term “cholinomimetic agent” refers to drugs that mimic the function of acetylcholine or enhance the activity of remaining acetylcholine synthesizing cells. Such drugs include, without limitation, inhibitors of acetylcholine degradation (acetylcholine esterase inhibitors, such as tacrine), drugs that mimic acetylcholine structure and function (e.g., muscarinic M1-receptor agonists), drugs that block acetylcholine uptake by neurons and drugs that interact pre-synaptic receptors to induce acetylcholine release from cholinergic neurons.

By “improving cognitive function” is meant to improve memory, learning capacity, communication language, thinking, decision making, judgment, and/or attention in a subject receiving treatment according to a method of the invention in comparison to the performance of the subject prior to receiving the treatment. The cognitive function can be evaluated using any of a variety of known tests for measuring and monitoring changes in cognitive function.

By “delaying the loss of cognitive function” is meant to delay the loss of memory, learning capacity, communication language, thinking, decision making, judgment, and/or attention in a subject receiving treatment according to a method of the invention in comparison to the average onset of loss of cognitive function observed for untreated subjects at the same stage of disease. The cognitive function can be evaluated using any of a variety of known tests for measuring and monitoring changes in cognitive function.

By “reducing the severity of an AD-associated neuropsychiatric condition” is meant to reduce one or more symptoms of an AD-associated neuropsychiatric condition, such as depression, anxiety, apathy, agitation, irritability, or aggression in comparison to the performance of the subject prior to receiving the treatment. The symptoms of the AD-associated neuropsychiatric condition can be evaluated using any of a variety of tests known in the art.

By “delaying the onset of an AD-associated neuropsychiatric condition” is meant to delay one or more symptoms of an AD-associated neuropsychiatric condition in a subject receiving treatment according to a method of the invention in comparison to the average onset of the AD-associated neuropsychiatric condition observed for untreated subjects at the same stage of disease. For example, the methods of the invention can be used to delay the onset of AD-associated depression and/or anxiety in a subject. The symptoms of the AD-associated neuropsychiatric condition can be evaluated using any of a variety of tests known in the art.

By “reducing the severity of dementia” is meant to reduce one or more symptoms of dementia in a subject receiving treatment according to a method of the invention in comparison to the performance of the subject prior to receiving the treatment. The symptoms of dementia can be evaluated using any of a variety of tests known in the art.

By “delaying the onset of dementia” is meant to delay one or more symptoms of dementia in a subject receiving treatment according to a method of the invention in comparison to the average onset of dementia observed for untreated subjects at the same stage of disease. The symptoms of dementia can be evaluated using any of a variety of tests known in the art.

As used herein, the term “treating” refers to administering treatment to a patient diagnosed with Alzheimer's disease (i) to ameliorate the disease and improve the patient's condition; or (ii) to delay the progression of Alzheimer's disease.

The term “unit dosage form” refers to physically discrete units suitable as unitary dosages, such as a pill, tablet, caplet, hard capsule or soft capsule, each unit containing a predetermined quantity of a lysergic acid diethylamide, or a pharmaceutically acceptable salt thereof. By “hard capsule” is meant a capsule that includes a membrane that forms a two-part, capsule-shaped, container capable of carrying a solid or liquid payload of drug and excipients. By “soft capsule” is meant a capsule molded into a single container carrying a liquid or semisolid payload of drug and excipients.

The term “extended release”, “controlled release” or “sustained release”, as used herein interchangeably, refers to a mode of releasing lysergic acid diethylamide, or a pharmaceutically acceptable salt thereof, from the formulation thereof such that it is absorbed by the body over a period of time and reducing the Cmax relative to that observed for administration of immediate release formulations administered at the same dosing level. An extended release formulation of an active agent may be accomplished, e.g., by embedding the active agent in a web of substance that the body is slow to dissolve, such that the active ingredient slowly and regularly leeches from the coating, or by swelling up the active agent to form a gel with a nearly impenetrable surface, wherein the drug slowly exits the semipermeable layer.

Other features and advantages of the invention will be apparent from the following Detailed Description and the claims.

### DETAILED DESCRIPTION

Therapy according to the invention may be performed alone or in conjunction with another therapy and may be provided at home or at a special purpose clinic, a hospital's outpatient department, or a hospital. The selected LSD dosing regimen for the therapy can depend on the age and condition of the patient, the stage and type of the subject's Alzheimer's disease, and how the subject responds to the treatment.

### Alzheimer's Disease

Alzheimer's disease is a devastating condition leading to progressive cognitive decline, functional impairment and loss of independence. It affects 5% of individuals over 65, 20% over 80 and more than a third of those over 90.

The symptoms of Alzheimer's disease are primarily marked by cognitive deficits including memory impairment, language dysfunction, and visuospatial skills; functional impairment that may span occupational and social issues (e.g., activities of daily living); and behavioral symptoms including depression, anxiety, aggression and psychosis may also appear as the disease progresses in severity.

The diagnosis of Alzheimer's disease can be based upon clinical findings of cognitive deficits consistent with AD and post-mortem identification of brain pathologies consistent with AD. The term AD dementia is used to describe dementia that is due to the pathophysiologies of Alzheimer's disease. There are currently a variety of art-accepted methods for diagnosing probable Alzheimer's disease. Typically, these methods can be used in combination. These methods include determining an individual's ability to carry out daily activities and identifying changes in behavior and personality. Dementia of the AD type is also typically characterized by an amnestic presentation (memory deficit) or language, visuospatial or executive function deficits. Cognitive ability/impairment may be determined by art-accepted methods, including, but not limited to, validated instruments that assess global cognition (e.g., the Modified Mini Mental State Examination (3MS-E)), and specific domains such as visual and verbal memory (e.g.,the Brief Visuospatial Memory Test (Revised) (BVMT-R) and the Hopkins Verbal Learning Test (Revised) (HVLT-R), respectively), language (e.g., the Generative Verbal Fluency Test (GVFT)), executive function and attention (e.g., the Digit Span Test (DST)), a Rey Auditory and Verbal Learning Test (RAVLT), Cambridge Neuropsychological Test Automated Battery (CANTAB), a Contextual Memory Test; a Continuous Recognition Memory Test (CMRT), a Denman Neuropsychology Memory Scale, a Fuld Object Memory Evaluation (FOME), a Graham-Kendall Memory for Designs Test; a Guild Memory Test; a Learning and Memory Battery (LAMB); a Memory Assessment Clinic Self-Rating Scale (MAC-S), a Memory Assessment Scales (MAS), a Randt Memory Test; a Recognition Memory Test (RMT), a Rivermead Behavioral Memory Test; a Russell's Version of the Wechsler Memory Scale (RWMS), a Test of Memory and Learning (TOMAL), a Vermont Memory Scale (VMS), a Wechsler Memory Scale, and a Wide Range Assessment of Memory and Learning (WRAML), First-Last Name Association (Youngjohn J. R., et al., Archives of Clinical Neuropsychology 6:287-300 (1991)), Name-Face Association, Wechsler Memory Scale-Revised; (Wechsler, D., Wechsler Memory Scale-Revised Manual, NY, N.Y., The Psychological Corp. (1987)), California Verbal Learning Test-Second Edition (Delis, D. C., et al., The Californian Verbal Learning Test, Second Edition, Adult Version, Manual, San Antonio, Tex.: The Psychological Corporation (2000)), Facial Recognition (delayed non-matching to sample), Cognitive Drug Research (CDR) Computerized Assessment Battery-Wesnes; Buschke's Selective Reminder Test (Buschke, H., et al., Neurology 24:1019-1025 (1974)), Telephone Dialing Test, and Brief Visuospatial Memory Test-Revised. Dementia due to AD is also defined by insidious onset and a history of worsening cognitive performance.

The course of the Alzheimer's disease can be divided into stages, with progressive patterns of cognitive and functional impairments. The stages include Mild/Early AD (typically lasting 2-4 years and characterized by frequent recent memory loss, particularly of recent conversations and events, mild coordination problems, depression and apathy accompanied by mood swings); Moderate/Middle AD (typically lasting 2-10 years and characterized by pervasive and persistent memory loss, including forgetfulness about personal history and inability to recognize friends and family, rambling speech, unusual reasoning, delusions, aggression, uninhibited behavior, slowness, rigidity, and tremors); and Severe/Late AD (typically lasting 1-3+ years and characterized by a loss of ability to remember, communicate, or process information, severe to total loss of verbal skills, problems with swallowing, incontinence, and illness, mood, behavior, hallucinations, and delirium).

Physicians may utilize a diagnostic framework with five, six, or seven levels. Progression through these stages usually lasts from 8 to 10 years, but can sometimes stretch out as long as 20 years. The seven stage Global Deterioration Scale, also known as the Reisberg Scale, includes the following dimensions.

Stage 1—No impairment. Memory and cognitive abilities appear normal.

Stage 2—Minimal Impairment/Normal Forgetfulness. Memory lapses and changes in thinking are rarely detected by friends, family, or medical personnel, especially as about half of all people over 65 begin noticing problems in concentration and word recall.

Stage 3—Early Confusional/Mild Cognitive Impairment. While subtle difficulties begin to impact function, the person may consciously or subconsciously try to cover up his or her problems. Subjects experience difficulty with retrieving words, planning, organization, misplacing objects, and forgetting recent learning, which can affect life at home and work. Depression and other changes in mood can also occur. Duration: 2 to 7 years.

Stage 4—Late Confusional/Mild Alzheimer's. Subjects experience problems handling finances result from mathematical challenges. Recent events and conversations are increasingly forgotten, although most people in this stage still know themselves and their family. Subjects experience problems carrying out sequential tasks, including cooking, driving, ordering food at restaurants, and shopping. Subjects often withdraw from social situations, become defensive, and deny problems.

Stage 5—Early Dementia/Moderate Alzheimer's disease. Decline is more severe and subject requires assistance. Subject is no longer able to manage independently or recall personal history details and contact information, and is frequently disoriented regarding place and or time. People in this stage experience a severe decline in numerical abilities and judgment skills, which can leave them vulnerable to scams and at risk from safety issues. Basic daily living tasks like eating and dressing require increased supervision.

Stage 6—Middle Dementia/Moderately Severe Alzheimer's disease. Subjects experience a total lack of awareness of present events and inability to accurately remember the past. People in this stage progressively lose the ability to take care of daily living activities like dressing, toileting, and eating, but are still able to respond to nonverbal stimuli, and communicate pleasure and pain via behavior. Agitation and hallucinations often show up in the late afternoon or evening. Dramatic personality changes, such as wandering or suspicion of family members are common. Many subjects can't remember close family members, but know they are familiar.

Stage 7—Late or Severe Dementia and Failure to Thrive. In this final stage, the subject's speech and ability to walk becomes severely limited. Total support around the clock is needed for all functions of daily living and care.

The methods and compositions of the invention can be used to reduce neuropsychiatric conditions associated with any of the stages of Alzheimer's disease described above. For example, the methods of the invention can reduce the severity of agitation, apathy, irritability, and aggression in a subject with Alzheimer's disease (i.e. a subject having AD at any of stages 2 to 7). The methods of the invention can delay the onset of agitation, apathy, irritability, and aggression in a subject with mild cognitive impairment due to Alzheimer's disease (i.e. a subject having AD at any of stages 2 to 4).

The methods and compositions of the invention can be used to treat any of the stages of Alzheimer's disease described above. For example, the methods of the invention can improve cognitive function or reduce the severity of dementia in a subject with Alzheimer's disease with comorbid dementia (i.e. a subject having AD at any of stages 5 to 7). The methods of the invention can delay the loss of cognitive function or delay the onset of dementia in a subject with mild cognitive impairment due to Alzheimer's disease (i.e. a subject having AD at any of stages 2 to 4).

The methods of the invention can be to delay the onset of cognitive impairment or delay the onset of dementia in a subject with asymptomatic Alzheimer's disease (i.e. a subject having AD at stage 1) or prodromal Alzheimer's disease (i.e. a subject having AD at stage 1 or 2 or 3).

The therapy of the invention can be used in combination with other therapeutic interventions for the treatment of Alzheimer's disease, such as ACE inhibitors or NMDA receptor antagonists, such as memantine.

### Formulation

For use in the methods and compositions of the invention, the lysergic acid diethylamide, or a pharmaceutically acceptable salt thereof, may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the lysergic acid diethylamide, or a pharmaceutically acceptable salt thereof, substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain drug action during a predetermined time period by maintaining a relatively, constant, effective drug level in the body with concomitant minimization of undesirable side effects associated with fluctuations in the plasma level of the lysergic acid diethylamide, or a pharmaceutically acceptable salt thereof (sawtooth kinetic pattern); (iv) formulations that localize drug action by, e.g., spatial placement of a controlled release composition adjacent to or in the diseased tissue or organ; and (v) formulations that target drug action by using carriers or chemical derivatives to deliver the lysergic acid diethylamide, or a pharmaceutically acceptable salt thereof, to a particular target cell type.

The methods of the invention can include administration of lysergic acid diethylamide, or a pharmaceutically acceptable salt thereof, in a dosage form designed for immediate release. Such immediate release formulations can include for administration intravenously, intramuscularly, or subcutaneously, orally, sublingually, by inhalation, or by topical or transdermal application. For example, the lysergic acid diethylamide, or a pharmaceutically acceptable salt thereof, can be formulated as a lozenge, drop, or film placed under the tongue for sublingual administration of a rapidly acting dosage form.

Administration of lysergic acid diethylamide, or a pharmaceutically acceptable salt thereof, in the form of a controlled release formulation is especially preferred in cases in which the subject receives a dosing regimen that at peak plasma levels can result in psychedelic side effects.

Any of a number of strategies can be pursued in order to obtain controlled release in which the rate of release outweighs the rate of metabolism of the lysergic acid diethylamide, or a pharmaceutically acceptable salt thereof. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes.

Solid Dosage Forms for Oral Use

Formulations for oral use include tablets containing the lysergic acid diethylamide, or a pharmaceutically acceptable salt thereof, in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the lysergic acid diethylamide, or a pharmaceutically acceptable salt thereof, in a predetermined pattern (e.g., in order to achieve a controlled release formulation) or it may be adapted not to release the lysergic acid diethylamide, or a pharmaceutically acceptable salt thereof, until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes. The coating may be applied on the solid dosage form in a similar manner as that described in Encyclopedia of Pharmaceutical Technology, supra.

Formulations for oral use may also be presented as chewable tablets, or as hard gelatin capsules wherein the lysergic acid diethylamide, or a pharmaceutically acceptable salt thereof, is mixed with an inert solid diluent (e.g., potato starch, lactose, microcrystalline cellulose, calcium carbonate, calcium phosphate or kaolin), or as soft gelatin capsules wherein the lysergic acid diethylamide, or a pharmaceutically acceptable salt thereof, is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may, e.g., be constructed to release the lysergic acid diethylamide, or a pharmaceutically acceptable salt thereof, by controlling the dissolution and/or the diffusion of the lysergic acid diethylamide, or a pharmaceutically acceptable salt thereof.

Dissolution or diffusion controlled release can be achieved by appropriate coating of a tablet, capsule, pellet, or granulate formulation of compounds, or by incorporating the lysergic acid diethylamide, or a pharmaceutically acceptable salt thereof, into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing the lysergic acid diethylamide, or a pharmaceutically acceptable salt thereof, may also be in the form of a buoyant tablet or capsule (i.e., a tablet or capsule that, upon oral administration, floats on top of the gastric content for a certain period of time). A buoyant tablet formulation of the lysergic acid diethylamide, or a pharmaceutically acceptable salt thereof, can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Liquids for Oral Administration

Powders, dispersible powders, or granules suitable for preparation of an aqueous suspension by addition of water are convenient dosage forms for oral administration. Formulation as a suspension provides the lysergic acid diethylamide, or a pharmaceutically acceptable salt thereof, in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

Parenteral Compositions

The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the lysergic acid diethylamide, or a pharmaceutically acceptable salt thereof, the composition may include suitable parenterally acceptable carriers and/or excipients. The lysergic acid diethylamide, or a pharmaceutically acceptable salt thereof, may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions according to the invention may be in the form suitable for sterile injection. To prepare such a composition, the lysergic acid diethylamide, or a pharmaceutically acceptable salt thereof, is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled Release Parenteral Compositions

Controlled release parenteral compositions may be in form of aqueous suspensions, microspheres, microcapsules, magnetic microspheres, oil solutions, oil suspensions, or emulsions. Alternatively, the lysergic acid diethylamide, or a pharmaceutically acceptable salt thereof, may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamnine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)).

Percutaneous and Topical Compositions

The pharmaceutical compositions may also be administered topically on the skin for percutaneous absorption in dosage forms or formulations containing conventionally non-toxic pharmaceutical acceptable carriers and excipients including microspheres and liposomes. The formulations include creams, ointments, lotions, liniments, gels, hydrogels, solutions, suspensions, sticks, sprays, pastes, plasters, and other kinds of transdermal drug delivery systems. The pharmaceutically acceptable carriers or excipients may include emulsifying agents, antioxidants, buffering agents, preservatives, humectants, penetration enhancers, chelating agents, gel-forming agents, ointment bases, perfumes, and skin protective agents.

Examples of emulsifying agents are naturally occurring gums (e.g., gum acacia or gum tragacanth) and naturally occurring phosphatides (e.g., soybean lecithin and sorbitan monooleate derivatives). Examples of antioxidants are butylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and AZONE™. Examples of chelating agents are sodium EDTA, citric acid, and phosphoric acid. Examples of gel forming agents are CARBOPOL™, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide (e.g., polyoxyethylene sorbitan monooleate (TWEEN™)).

The pharmaceutical compositions described above for topical administration on the skin may also be used in connection with topical administration onto or close to the part of the body that is to be treated. The compositions may be adapted for direct application or for introduction into relevant orifice(s) of the body (e.g., rectal, urethral, vaginal or oral orifices). The composition may be applied by means of special drug delivery devices such as dressings or alternatively plasters, pads, sponges, strips, or other forms of suitable flexible material.

Controlled Release Percutaneous and Topical Compositions

There are several approaches for providing rate control over the release and transdermal permeation of a drug, including: membrane-moderated systems, adhesive diffusion-controlled systems, matrix dispersion-type systems, and microreservoir systems. A controlled release percutaneous and/or topical composition may be obtained by using a suitable mixture of the above-mentioned approaches.

In a membrane-moderated system, the lysergic acid diethylamide, or a pharmaceutically acceptable salt thereof, is present in a reservoir which is totally encapsulated in a shallow compartment molded from a drug-impermeable laminate, such as a metallic plastic laminate, and a rate-controlling polymeric membrane such as a microporous or a non-porous polymeric membrane (e.g., ethylene-vinyl acetate copolymer). The lysergic acid diethylamide, or a pharmaceutically acceptable salt thereof, is only released through the rate-controlling polymeric membrane. In the drug reservoir, the lysergic acid diethylamide, or a pharmaceutically acceptable salt thereof, substance may either be dispersed in a solid polymer matrix or suspended in a viscous liquid medium such as silicone fluid. On the external surface of the polymeric membrane, a thin layer of an adhesive polymer is applied to achieve an intimate contact of the transdermal system with the skin surface. The adhesive polymer is preferably a hypoallergenic polymer that is compatible with the drug.

In an adhesive diffusion-controlled system, a reservoir of the lysergic acid diethylamide, or a pharmaceutically acceptable salt thereof, is formed by directly dispersing the drug in an adhesive polymer and then spreading the adhesive containing the drug onto a flat sheet of substantially drug-impermeable metallic plastic backing to form a thin drug reservoir layer. A matrix dispersion-type system is characterized in that a reservoir of the drug substance is formed by substantially homogeneously dispersing the drug substance in a hydrophilic or lipophilic polymer matrix and then molding the drug-containing polymer into a disc with a substantially well-defined surface area and thickness. The adhesive polymer is spread along the circumference to form a strip of adhesive around the disc.

In a microreservoir system, the reservoir of the lysergic acid diethylamide, or a pharmaceutically acceptable salt thereof, is formed by first suspending the drug solid in an aqueous solution of water-soluble polymer, and then dispersing the drug suspension in a lipophilic polymer to form a plurality of microscopic spheres of drug reservoirs.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLE 1

### Immediate Release Capsules

D-lysergic acid diethylamide tartrate is mixed with pharmaceutically suitable diluents (e.g., talc, silica, lactose) and placed into gelatin capsules. Formulated for immediate release, LSD's effects can typically last from 6-12 hours depending on dosage, tolerance, body weight and age. Immediate release LSD dosed at 1 μg/kg can have an apparent plasma half-life of 5.1 hours, with a peak plasma concentration of 5 ng/mL at 3 hours post-dose.

Capsules containing 5 μg, 10 μg, 15 μg, and 20 μg D-lysergic acid diethylamide tartrate can be useful in the methods of the invention.

### EXAMPLE 2

### Sustained Release Pellets

Povidone USP (PVP K29/32) is dissolved in distilled water and ethanol 96% mixture, and D-lysergic acid diethylamide tartrate is dissolved in the formed solution. Talc extra fine is dispersed into the solution to form a uniform suspension, which is then coated onto sugar spheres of 600-710 μm using a fluid bed coater. In a separate container, a functional coating suspension is prepared by mixing Ethocel 45 cps (ethylcellulose; a release control polymer) in acetone and ethanol 96% mixture with polyethylene glycol (PEG) 4000 dissolved in distilled water to the form a coating mixture. The coating mixture is then coated onto the LSD-loaded pellets using a fluid bed coater.

The sustained release pellets permit D-lysergic acid diethylamide to be released slowly following oral administration such that the maximum circulating concentration (Cmax) is reduced relative to the Cmax observed for the immediate release formulation of Example 1 administered at the same dosing level. Capsules containing 5 μg, 10 μg, 15 μg, and 20 μg D-lysergic acid diethylamide tartrate can be useful in the methods of the invention.

### EXAMPLE 3

### Evaluation of the Efficacy of Lysergic Acid Diethylamide in a Transgenic Rat Model of Alzheimer's Disease.

Female and male 6-7-week old homozygous McGill-R-Thy1-APP transgenic (Tg) AD rats and their littermate wildtype controls (WT) rats (see Hanzel et al., Neurobiology of aging 35:10 2249 (2014); and Leon et al., Journal of Alzheimer's Disease 20:1 113 (2010)) are housed in rat standard cages in mixed-genotype and treatment groups of 2 animals and maintained on a 12/12 light/dark cycle. The room temperature is maintained between 20 and 23° C. with a relative humidity maintained between 30% and 70%. Standard rodent chow and water is provided ad libitum. Rats are examined and handled for one week prior to initiation of the study to assure adequate health and suitability and to minimize non-specific stress associated with manipulation.

Lysergic acid diethylamide (LSD) is dissolved in saline and administered intraperitoneally for 4 months at a dose volume of 1 ml/kg in a dosing schedule of every 3rd day (i.e., 2 days between dosings) at the beginning of the dark period, starting when rats are 8 weeks of age.

Pathology Analysis

Tissues from all AD Tg rats are collected for analysis. Tissues from WT rats are collected to serve as controls. At the end of behavioral study, brains are collected from all rats. Briefly, rats are deeply anesthetized with Nembutal (60 mg/kg, i.p.) and perfused for 1 minute with ice-cold saline solution (pH 7.4). The brain is quickly removed and separated into right and left hemispheres on ice. Each hemisphere is treated as described below.

Immunohistochemistry

Immunohistochemistry is performed with right hemibrains of the rat model. The right hemisphere is prepared by immersion-fixed in 4% paraformaldehyde in 0.1 M phosphate buffer (pH 7.4) for 24 hours at 4° C. Astrocyte activation and microglia are measured by GFAP and Iba-1 staining in the regions of interest (cortex, CA1, CA2, CA3 and subiculum). Each brain hemisphere is cut at 40 μm coronal sections through the hippocampal region and take two sections from one level. The sections are stained with GFAP for activated astrocytes and Iba-1 for microglia. Exact Bregma level is determined. The stained sections are imaged on a Mirax whole slide scanner and quantification of GFAP and Iba-1 density will be performed. Quantitative assessment is made of GFAP, including estimation of density, in cortex, CA1, CA2, CA3, and subiculum in rat hemibrains Quantitative assessment of Iba-1, including estimation of density, in cortex, CA1, CA2, CA3, and subiculum in rat hemibrains.

Biochemistry

Biochemistry is performed in cortex and hippocampus hemibrains of the rat model. The left hemisphere of the brain is dissected to separate cortex and hippocampus. These brain areas are snap-frozen in liquid nitrogen and stored at −80° C. until biochemical analyses are performed.

Cortex and hippocampus samples are homogenized for about 30 seconds in short bursts with tissue homogenizer (Brinkmann) in 1 mL of 1× TBS-buffer containing protease inhibitor per 100 mg of tissue. Care is taken to keep the tissue from over heating. Tissue homogenate is split into two parts, part one is used for quantification of soluble and insoluble Aβ 40/42 (following manufacturer's protocol) and part two will be used for quantification of COX-2 (ELISA) and other inflammatory markers by Luminex, following manufacturer's protocol.

Quantification of soluble and insoluble Aβ 40/42 in Brain using ELISA: tissue homogenates are used according to manufacturer's protocol to measure levels of soluble and insoluble levels of Aβ 40/42 in cortex and hippocampus of rat brains (ELISA kits from Millipore EMD; St. Charles, Mo.).

Quantification of rat COX-2(cyclooxygenase-2) in Brain using ELISA:

Levels of rat COX-2 are measured in cortex and hippocampus using ELISA (IBL International; Toronto, Canada). Tissue homogenates are centrifuged for 30 minutes at 4° C., lysates are diluted 1:10 with appropriate matrix provided with kit.

Inflammatory Markers Levels Assessment with LuminexMAP®: inflammatory marker levels in cortex and hippocampus are measured with Luminex Technology using Bio-Plex MAP kit (Bio-Rad Laboratories; Hercules, Calif.). Briefly, sample homogenates are centrifuged for 30 minutes at 4° C., lysates are diluted 1:4 with appropriate matrix provided with kit, followed by incubation with a specific color-coded magnetic bead coated with an inflammatory marker-specific antibody. After the analyte is captured by the bead, a biotinylated detection antibody is introduced. The reaction mixture is then incubated with streptavidin-PE conjugate, a reporter molecule, to complete reaction on the surface of the microsphere bead. Microsphere beads are then excited by light-emitting diodes (LEDs) and detected with CCD camera. Mean fluorescent intensity (MFI) is quantified based on a standard curved. The following inflammatory marker levels (pg/mL) are measured: TNF-alpha, IL-6, IL-1beta, IL-4, IL-10, IL-13, and TGF-β (isoforms 1, 2, and 3).

Statistical Analysis

Data are analyzed by analysis of variance (ANOVA) followed by post-hoc comparisons where appropriate. Data are represented as the mean and standard error to the mean (s.e.m). This study can show that treatment with lysergic acid diethylamide, or a salt thereof, is capable of (i) reducing the severity of one or more symptoms of Alzheimer's disease, or (ii) delaying the onset of one or more symptoms of Alzheimer's disease. Another benefit of treatment with lysergic acid diethylamide, or a salt thereof, is a normalization, or delay in the typical disease progression, of the underlying biomolecular markers characteristic of Alzheimer's disease.

### EXAMPLE 4

### Protocol for a Phase 2 Clinical Study to Evaluate Safety, Tolerability, and Efficacy of LSD for Individuals with Mild Cognitive Impairment due to Alzheimer's Disease.

The study is a Bayesian adaptive randomization design study for evaluating the safety, tolerability and efficacy of LSD on individuals with MCI due to AD. It includes an interim analyses (IAs) to adjust the number of subjects in the trial and the trial duration.

Participants are selected upon their fulfilment of the NIA-AA criteria for the diagnosis of MCI due to AD—intermediate likelihood, at the clinical screening visit. Other inclusion criteria may include the following: (i) must have a positive amyloid PET scan and positive CSF Aβ₄₂ or CSF Tau assessment; (ii) must be between the age of 50 and 90 years, inclusive; (iii) must report a history of subjective memory decline with gradual onset and slow progression over the last one year before screening and must be corroborated by an informant; (iv) must have been on a stable medication regime for more than 3 months prior to screening; (v) must have a minimum of 7 years of formal education; and/or (vi) must have adequate visual and auditory acuity to allow neuropsychological testing based on the research clinician's judgment.

Exclusion criterial may include the following: (i) any neurological condition that may be contributing to cognitive impairment above and beyond that caused by the patient's AD; (ii) history of transient ischemic attacks (TIA), stroke, or seizures within 12 months of screening; (iii) contraindications to MRI scanning, including cardiac pacemaker/defibrillator, ferromagnetic metal implants, e,g., in skull and cardiac devices other than those approved as safe for use in MR scanners; (iv) evidence of other clinically significant lesions that could indicate a dementia diagnosis other than AD on brain MRI at Screening, or other significant pathological findings on brain MRI at Screening; (v) a patient with the lifetime presence of any of the following is excluded: psychotic symptom that is not substance-induced or due to a medical condition; any manic or hypomanic episode; (vi) patient is receiving chronic administration of tricyclic antidepressants or lithium or acute administration of serotonin reuptake inhibitors or haloperidol, or serotonin-norepinephrine reuptake inhibitors or monoamine oxidase inhibitors; and/or (vii) patient is taking OTC doses of 5-hydroxytryptophan or St John's Wort or Ayahuasca (which contains monoamine oxidase inhibitors in addition to dimethyltryptamine).

The study includes a screening period of up to eight weeks. Following provision of informed consent and completion of all screening assessments, eligible subjects are assigned on a randomized basis to one of the two treatment groups (Group #1 and Group #2). Following the completion of a baseline evaluation (Visit #3), subjects in Group #1 receive from 2 to 30 μg (e.g., 25±5, 15±5 μg, 12.5±5 μg, 10±2 μg, 8±2 μg, 7.5±2.5 μg, 6±2 μg, or 4±2 μg of lysergic acid diethylamide or a pharmaceutically acceptable salt thereof) administered P.O. or S.C., while Group #2 receive placebo. Thereafter, dosing for Group #1 and #2 would occur every fourth day for the duration of the study. During screening, baseline, and treatment days, subjects undergo a series of assessments as outlined in the schedule.

The study includes an eight-week clinical and imaging screening period with drug administration once every four days, with a maximum treatment period of 104 weeks. IAs can be scheduled at intervals algorithmically determined by the study's recruitment/randomization rate and the number of patient treatment days, subject to the early termination of the study based upon predetermined futility or success IA signals.

The primary objective of the study is an observation of a change from baseline in a modified Alzheimer's Disease Cooperative Study—Preclinical Alzheimer Cognitive Composite (ADCS-PACC) battery or a similar measure of cognitive function, optionally with one or more secondary objectives. Secondary objectives include observation of changes in microglial activation on [11C] (R)-PK-111-95 PET scans from baseline; observation in change in cortical amyloid load via Positron Emission Tomography (PET) scan from screening; observation in changes in Volumetric Magnetic Resonance Imaging (vMRI) from baseline; observation of Cognitive Function Index changes from baseline; observation in change of the Apathy Inventory from baseline; observation of changes in Cerebral Spinal Fluid (CSF) biomarkers of Tau, Amyloid Beta (Aβ 42), and Inflammatory Biomarkers (IL-1, IL-6, TNF-α, IFN-γ, IL-4, IL-10, IL-12, IL-13, MCP-1, GM-CSF, YKL-40, CRP, TGF-β); observation in change in the levels of plasma markers of neuroinflammation (IL-1, IL-6, TNF-α, IFN-γ, IL-4, IL-10, IL-12, IL-13, MCP-1, GM-CSF, YKL-40, CRP, TGF-β); assessment of pharmacodynamics effects of treatment, including mood, cognition, and other affective measures.

Safety evaluations are performed by monitoring adverse events (AEs), vital signs, 12-lead electrocardiograms (ECGs), standard clinical laboratory safety tests, physical examination findings, and suicidality evaluation using the Columbia-Suicidal Severity Rating Scale (C-SSRS) during study visits.

Statistical analysis is performed using standard methods.

One or more symptoms of Alzheimer's disease are ameliorated by administration of a psychedelic compound, such as LSD, using the methods described herein. For example, the treatment can include administering from 2 to 30 μg (e.g., 25±5, 15±5 μg, 12.5±5 μg, 10±2 μg, 8±2 μg, 7.5±2.5 μg, 6±2 μg, or 4±2 μg of lysergic acid diethylamide or a pharmaceutically acceptable salt thereof) to a subject with Alzheimer's disease, as described in Example 4.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method of treating Alzheimer's disease in a subject, said method comprising administering to the subject a pharmaceutical composition comprising lysergic acid diethylamide, or a pharmaceutically acceptable salt thereof, in an amount sufficient to treat said Alzheimer's disease.

2. The method of claim 1, wherein said pharmaceutical composition is a unit dosage form comprising from 2 to 30 μg of lysergic acid diethylamide or a pharmaceutically acceptable salt thereof.

3. The method of claim 2, wherein said pharmaceutical composition is a unit dosage form comprising 10±2 μg of lysergic acid diethylamide or a pharmaceutically acceptable salt thereof.

4. The method of any one of claims 1-3, comprising improving cognitive function, reducing the severity of an AD-associated neuropsychiatric condition, delaying the loss of cognitive function, or delaying the onset of an AD-associated neuropsychiatric condition in said subject.

5. The method of claim 4, comprising reducing agitation, reducing irritability, reducing apathy, or reducing aggression in said subject.

6. The method of claim 4, comprising delaying the onset of agitation, irritability, apathy, or aggression in said subject.

7. The method of claim 4, comprising improving memory in said subject; improving learning capacity in said subject; or delaying the loss of memory in said subject.

8. The method of claim 4, comprising delaying the loss of learning capacity in said subject.

9. The method of claim 4, comprising reducing the severity of dementia in said subject or delaying the onset of dementia in said subject.

10. The method of claim 4, comprising reducing the severity of depression in said subject or delaying the onset of depression in said subject.

11. The method of claim 4, comprising reducing the severity of anxiety in said subject or delaying the onset of anxiety in said subject.

12. The method of any one of claims 1-3, comprising reducing agitation, reducing apathy, reducing irritability, or reducing aggression in a subject having Alzheimer's disease with comorbid dementia.

13. The method of any one of claims 1-3, comprising reducing agitation, reducing apathy, reducing irritability, or reducing aggression in a subject having Alzheimer's disease with mild cognitive impairment due to Alzheimer's disease.

14. The method of any one of claims 1-3, comprising reducing agitation, reducing apathy, reducing irritability, or reducing aggression in a subject having Alzheimer's disease with asymptomatic Alzheimer's disease.

15. The method of any one of claims 1-3, comprising reducing agitation, reducing apathy, reducing irritability, or reducing aggression in a subject having Alzheimer's disease with prodromal Alzheimer's disease.

16. The method of any one of claims 1-3, comprising improving cognitive function or reducing the severity of dementia in a subject having Alzheimer's disease with comorbid dementia.

17. The method of any one of claims 1-3, comprising improving cognitive function or reducing the severity of depression in a subject having Alzheimer's disease with comorbid depression.

18. The method of any one of claims 1-3, comprising improving cognitive function or reducing the severity of anxiety in a subject having Alzheimer's disease with comorbid anxiety.

19. The method of any one of claims 1-3, comprising delaying the loss of cognitive function or reducing the severity of an AD-associated neuropsychiatric condition in a subject having Alzheimer's disease with comorbid dementia.

20. The method of any one of claims 1-3, comprising delaying the loss of cognitive function or delaying the onset of an AD-associated neuropsychiatric condition in a subject with mild cognitive impairment due to Alzheimer's disease.

21. The method of any one of claims 1-3, comprising delaying the loss of cognitive function or delaying the onset of an AD-associated neuropsychiatric condition in a subject with asymptomatic Alzheimer's disease.

22. The method of any one of claims 1-3, comprising delaying the loss of cognitive function or delaying the onset of an AD-associated neuropsychiatric condition in a subject with prodromal Alzheimer's disease.

23. The method of any one of claims 19-22, wherein said AD-associated neuropsychiatric condition is depression and/or anxiety.

24. The method of any one of claims 19-22, wherein said AD-associated neuropsychiatric condition is agitation.

25. The method of any one of claims 19-22, wherein said AD-associated neuropsychiatric condition is apathy.

26. The method of any one of claims 19-22, wherein said AD-associated neuropsychiatric condition is aggression.

27. The method of any one of claims 19-22, wherein said AD-associated neuropsychiatric condition is irritability.

28. The method of any one of claims 19-22, wherein said cognitive function is memory and/or learning capacity.

29. The method of any one of claims 1-28, wherein said lysergic acid diethylamide, or a pharmaceutically acceptable salt thereof, is administered in a dosing regimen from once daily to once weekly.

30. The method of claim 29, wherein said dosing regimen is once every three, four, or five days.

31. The method of claim 29 or 30, wherein said dosing regimen comprises administering to said subject an average of from 8 μg to 90 μg lysergic acid diethylamide, or a pharmaceutically acceptable salt thereof, per week.

32. The method of any one of claims 1-31, wherein said pharmaceutical composition is formulated for sustained release.

33. The method of any one of claims 1-31, wherein said pharmaceutical composition is formulated for immediate release.

34. The method of claim 1, further comprising administering to said subject a neuronal growth factor, a neuronal survival factor, a neuronal trophic factor, a cholinergic modulator, an adrenergic modulator, a nonadrenergic modulator, a dopaminergic modulator, a glutaminergic modulator or an agent that modulates PKC, PKA, GABA, NMDA, cannabinoid, AMPA, kainite modulator, phosphodiesterase (PDE), CREB or nootropic pathways within 1-30 days of administering said lysergic acid diethylamide, or a pharmaceutically acceptable salt thereof.

35. The method of claim 1, further comprising administering to said subject a cholinomimetic agent within 1-30 days of administering said lysergic acid diethylamide, or a pharmaceutically acceptable salt thereof.

36. The method of claim 35, wherein said cholinomimetic agent is selected from inhibitors of acetylcholine degradation, inducers of acetylcholine synthesis, acetylcholine agonists, and muscarinic M2-receptor antagonists.

37. The method of claim 36, wherein said cholinomimetic agent is an acetylcholinesterase inhibitor.

38. The method of claim 1, further comprising administering to said subject an NMDA antagonist within 1-30 days of administering said lysergic acid diethylamide, or a pharmaceutically acceptable salt thereof.

39. The method of claim 38, wherein said NMDA antagonist is memantine, or a pharmaceutically acceptable salt thereof.
